(19) United States
(12) Patent Application Publication
(10) Pub. No.: US 2013/0317419 A1
(43) Pub. Date: Nov. 28, 2013

(54) ENDOSCOPIC SURGERY INSTRUMENTATION

(71) Applicant: James K. Brannon, Leawood, KS (US)

(72) Inventor: James K. Brannon, Leawood, KS (US)

(21) Appl. No.: 13/944,696

(22) Filed: Jul. 17, 2013

Related U.S. Application Data
(62) Division of application No. 12/908,879, filed on Oct. 21, 2010, now abandoned.

(51) Int. Cl. A61M 3/02; A61M 39/22; A61M 1/00

(52) U.S. Cl. CPC A61M 3/0283; A61M 3/02; A61M 1/0031; A61M 39/22
USPC 604/28; 604/256; 604/500; 606/49

(57) ABSTRACT

An embodiment of the invention includes a quantum fluid flow management apparatus which controls and facilitates delivery of liquid and/or air to the surgical site during a surgical procedure via a trumpet valve, various restriction (crimping) devices, and a network of tubing for selectively passing air or fluid through to the surgical site. The trumpet valve allows for selective control of air or fluid or both or neither. Additionally the flow management apparatus controls the application and suction of fluids from the surgical site which may be used in cooperation with the injection of a fluid such as water to irrigate the surgical site to remove body fluids, debrided material or the like.

### CROSS-REFERENCE TO RELATED APPLICATION

This application is a divisional application claiming the benefit of the prior filed U.S. non provisional application Ser. No. 12/908,879, filed Oct. 21, 2010, which claims the benefit of the prior filed U.S. provisional application No. 61/253,068 filed Oct. 20, 2009 which is incorporated herein by reference.

### BACKGROUND OF THE INVENTION

The present invention is broadly concerned improvements in instruments for endoscopic surgery.

Modern surgery tends toward minimally invasive techniques whenever possible. Although often more complicated in some ways for the surgeon, minimally invasive techniques result in less trauma to the patient and less scarring because of much smaller incisions thereby promoting faster healing and reducing possibilities for infections. In general, minimally invasive surgeries involve making one or more small incisions at appropriate locations and inserting tubular devices through the incisions to the surgical site. The tubular devices may be referred to as endoscopes, arthroscopes, and the like and typically have optical fiber based optical viewing apparatus and light sources, surgical instruments, lumens for exchanging fluids with the surgical site, or combinations thereof extending therethrough. In some circumstances it is more appropriate to separate the light source and viewing scope from specifically surgical instruments, thus requiring two incisions and endoscopes. This technique is sometimes referred to as triangulation. In other instances, external types of imaging techniques are used for locating endoscopic instruments, such as fluoroscopes, computed tomography, magnetic resonance imaging, or the like.

Endoscopic instruments are configured in a number of different ways depending on their intended purpose. There are rigid endoscopes and flexible endoscopes. Some are simply tubes through which provide access to a surgical site for instruments which are passed through the scopes or for the exchange of fluids to and from the surgical site. Viewing scopes, including light sources, may be used for viewing a surgical site for diagnostic purposes or to view surgical operations occurring through the same scope or a different scope. Surgical operations may include cutting, shaving, debriding, cauterizing, or the like as well as grasping tissues or parts of organs, such as with forceps.

### SUMMARY OF THE INVENTION

The present invention provides improvements in certain areas of minimally invasive surgical techniques and instrumentation.

An embodiment of the invention includes a quantum fluid flow management apparatus which controls and facilitates delivery of liquid and/or air to the surgical site during a surgical procedure via a trumpet valve, various restriction (crimping) devices, and a network of tubing for selectively passing air or fluid through to the surgical site. The trumpet valve allows for selective control of air or fluid or both or neither. Additionally the flow management apparatus controls the application and suction of fluids from the surgical site which may be used in cooperation with the injection of a fluid such as water to irrigate the surgical site to remove body fluids, debrided material or the like.

An embodiment of the invention includes an improved trephine which includes a shaped surface with a stopper at the end with an opening at a front, a rear, and a side entry for the passage of surgical instruments including flexible forceps, passage of fluid/air and providing a resealable rear entry for containing any fluid/air within the surgical site. The trephine is adapted for receiving a shaver or other instrument with a rear portal opening having a shaped surface for which the new trephine is adapted for providing a hermetic seal thereabout and the trephine includes a shaped end which is adapted for hermetic sealing the end of the trephine. The trephine also allows for the receipt of standard surgical instruments including the flexible forceps.

The tip of the trephine may be formed to present a cutting edge providing a cutting instrument when in the receipt of a shaver and the tip disposed within the patient near the surgical site may also be curved or straight for passing the flexible forceps through the ligament head into the femoral head with around a 30° curve. The tip of the end may also be removable for passage of the forceps as needed. The trephine may be telescoping as desired to substantially place the tip below a fluid level.

An embodiment of the improved trephine is provided with a conically tapered distal tip of the cannula which is then cut at an angle. The tapering allows the cannula to pass more easily through the incision to the surgical site with reduced incidence of injury to intervening tissues.

An embodiment of the improved trephine is provided with a visual indicator of the direction of the angled cut tip of the cannula. A side port of the trephine is secured to a ring which is rotatable relative to the body of the trephine. Passages are provided within either the ring of the body of the trephine to provide fluid communication between the side port and the main passage of the trephine body. An indicator post projects from the proximal end of the body and shows the rotated position of the body, and thus the angular position of the tip, with respect to the side port. The indicator post may also facilitate rotation of the body and cannula.

In addition, the trephine may be used as a Boveri device for electro-cauterization of the surgical site as needed by associating an electric charge with the invention presenting a cauterization tip for treatment with the trephine presenting the proper electric connectivity for passing the electrical charge down to the tip of the flexible forceps.

Another embodiment of the invention includes an improved outflow adapter which is provided for use with other types of endoscopic instruments, such as an arthroscopic shaver, to provide triangular outflow from a surgical site. The outflow adapter includes an adapter body with a proximal end port, a distal end port, and a side or lateral port. A pair of plugs, including a closed plug and an open plug, are provided for insertion into the proximal end port to either close the proximal end or to allow outflow therefrom. The distal end of the outflow adapter is sized to be sealingly received in a proximal end of the endoscopic instrument with which it is used.

Various objects and advantages of the present invention will become apparent from the following description taken in conjunction with the accompanying drawings wherein are set forth, by way of illustration and example, certain embodiments of this invention.

The drawings constitute a part of this specification, include exemplary embodiments of the present invention, and illustrate various objects and features thereof.

### BRIEF DESCRIPTION OF THE DRAWINGS

FIG. 1 is a view of an embodiment of quantum flow fluid management apparatus according to the present invention.

FIG. 2 is a view similar to FIG. 1 and shows the fluid management apparatus of the present invention.

FIG. 3 is a block diagram illustrating components of the fluid management apparatus of the present invention.

FIG. 4 is an enlarged photograph of a trumpet valve unit of the fluid management apparatus.

FIG. 5 is a photographic side view of an embodiment of an improved trephine according to the present invention.

FIG. 5A is an enlarged fragmentary side elevational view of a distal end of an embodiment of the improved trephine and illustrates a conical tapering of the distal end.

FIG. 6 is a photographic perspective view of the improved trephine from a proximal end and illustrates a visual indicator in a non-aligned angular relationship with a side port of the trephine.

FIG. 7 is an enlarged photographic side view of the embodiment of the improved trephine with a portion broken away to illustrate exemplary details thereof.

FIG. 8 is an enlarged photographic side view of an embodiment of an outflow adapter according to the present invention along with a pair of stoppers for use with the outflow adapter.

FIG. 9 is an enlarged photographic perspective view of the outflow adapter and illustrates embodiments of closed and open stoppers for use with the outflow adapter.

FIG. 10 is an enlarged photographic perspective view of the outflow adapter with the closed stopper inserted into a proximal end thereof.

FIG. 11 is a view similar to FIG. 10 and shows the outflow adapter with the open stopper inserted into the proximal end thereof.

FIG. 12 is a photographic side view of the outflow adapter shown inserted into a proximal end of an elongated endoscopic surgical instrument.

FIG. 13 is a photographic side view of the outflow adapter shown inserted into the proximal end of a short endoscopic surgical instrument.

FIG. 14 is a greatly enlarged photographic side view of the outflow adapter shown with the open stopper inserted and shown inserted into the proximal end of an alternative configuration of an endoscopic surgical instrument.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to the drawings in more detail, the reference numeral 1 generally designates an embodiment of a quantum flow or fluid management apparatus according to the present invention. In general, the fluid management apparatus 1 controls the flow of fluids such as water or gases such as air to a surgical site associated with a patient and the removal of fluids or gases from the surgical site, either by release and outflow thereof or by the use of suction.

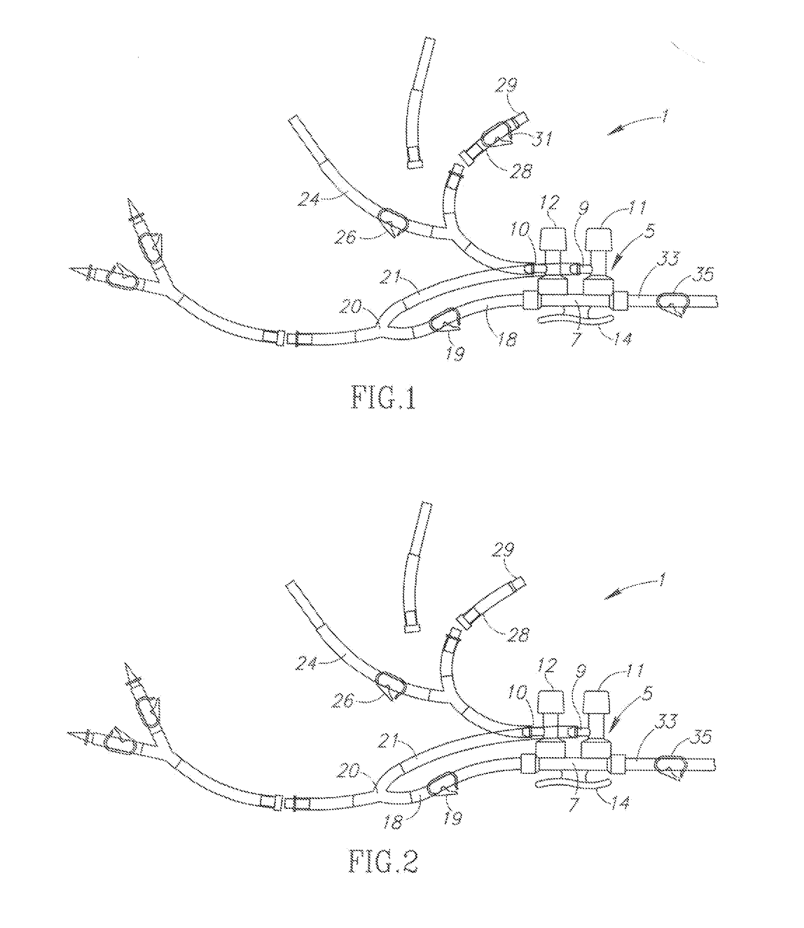

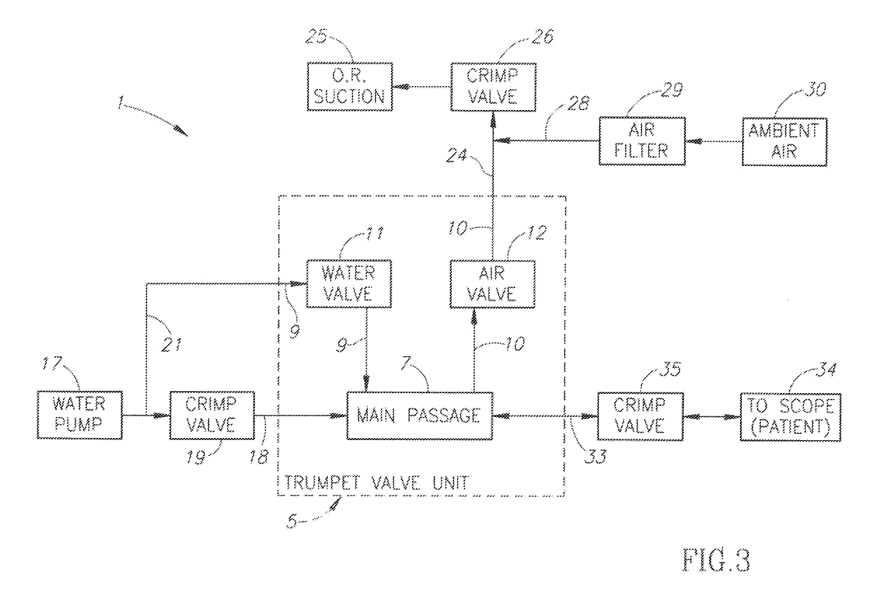

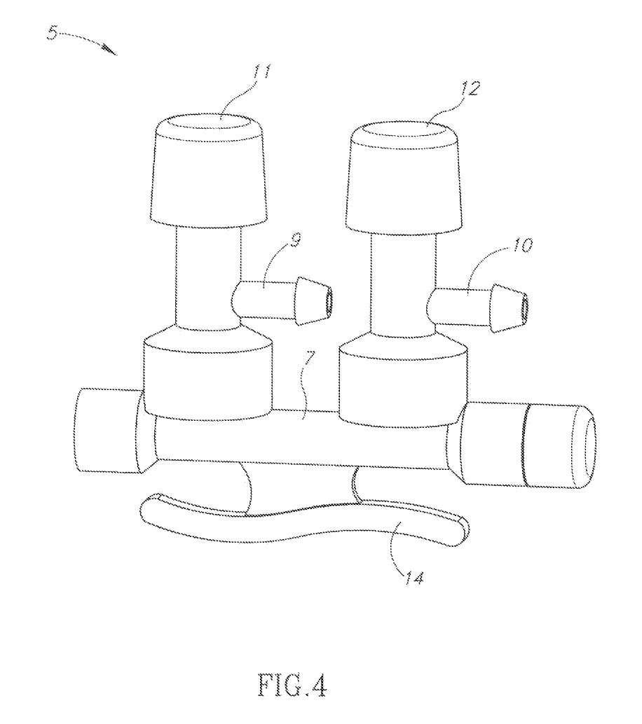

Referring to FIGS. 1-4, the illustrated fluid management apparatus 1 includes a trumpet valve unit 5 including a main passage 7 (FIG. 4), and a pair of branch passages 9 and 10. The trumpet valve unit 5 includes valves 11 and 12 which control fluid communication respectively between the branch passages 9 and 10 and the main passage 7. The valves 11 and 12 are normally closed and are manually opened against spring pressure to provide fluid communication therethrough. As depicted the valve 11 is associated with a fluid or hydraulic source and the valve 12 is associated with a pneumatic source, although the valves may be arranged alternatively as desired. The trumpet valve unit 5 includes a grip member 14 positioned on the tubular member forming the main passage 7 to facilitate grasping the unit 5 and operating the valves 11 and 12 by a surgeon or a surgical assistant during a surgical procedure.

In the illustrated embodiment of the fluid management apparatus 1, a fluid source, pump or other fluid delivery means 17 is connected through a main fluid conduit 18 through a removable crimp valve 19 to the main passage 7 of the trumpet valve unit 5. A Y-branch member 20 is positioned in the main fluid conduit 18 and connects the conduit 18 to the fluid branch passage 9 by way of a branch fluid conduit 21. The crimp valve 19 is of the ratcheted type and is operable to pinch or crimp the main fluid conduit 18 to control flow therethrough. The crimp valve 19 can be released or removed to enable continuous flow of fluid through the main passage 7. As illustrated, the fluid source 17 may be paired via a Y-branch fluid supply line 16 to facilitate addition or removal of fluid during the surgical procedure. In addition, the supply line 16 may be coupled to the Y-branch member 20 via easy coupling connectors 38, with a female and male portions 38a, 38b. Generally, the easy coupling connectors facilitate rapid convenient attachment of the fluid to the fluid management apparatus 1.

A suction conduit 24 connects an operating room suction source 25 to the branch passage 10 of the trumpet valve unit 5 through a crimp valve 26. Alternatively, an air supply connector 32 may be utilized through a connector coupling or as illustrated by a Y-branch member 27 in the conduit 24 with an air conduit 28 connected to it. An air filter 29 can be positioned at an end of the air conduit 28 to filter ambient air 30 entering the air conduit. The air conduit 28 may also have an air crimp valve 31 positioned on it. The crimp valves 19, 26, and 31 can be partially applied through an adjustable ratchet to restrict flow as desired at their respective conduits, partially or fully applied to completely close the conduit.

The illustrated fluid management apparatus 1 includes a main conduit or patient conduit 33 connected to the main passage 7 and to an endoscopic instrument 34 at the surgical site through a crimp valve 35.

The fluid management apparatus 1 enables the surgeon to alternately and repeatedly depress the water valve 11 to inject water or other fluid to the surgical scope 34 at the surgical site or to aspirate the fluid or other matter from the surgical site by selectively pressing the air or suction valve 12. These operations can be performed to initially clean up the surgical site or to remove tissue, debrided matter, or bodily fluids from the surgical site as the procedure is performed. The configuration of the apparatus 1, including the trumpet valve 5 and the length of conduits 18, 24, and 33, enables the surgeon to stretch his or her arms, with one hand on the trumpet valve unit 5, the other hand on an endoscopic instrument 34, and eyes on a video monitor (not shown) to achieve a more balanced and comfortable stance to thereby reduce fatigue during surgery.

Alternatively, two scopes may be interconnected by the fluid management apparatus in a triangulation configuration with one scope attached to the main patient conduit 33 and another scope or other surgical instrument attached to the triangulation scope conduit via an alternative air supply conduit 32 connectably secured at the crimp valve 26. In this manner one or more surgical instruments may be introduced into the cavity through multiple scopes at different openings with the distal ends of each scope being angularly oriented to the surgical site. By connecting one scope to the air branch passage 10 and one scope to the patient conduit 33 the valves 11 and 12 can be operated simultaneously to provide a steady stream of irrigating fluid to the surgical site which is continuously drawn from the site by the suction source 25. The crimp valves 19, 26, and 35 can be adjusted by the surgeon to control the flow rate of fluid to the surgical site and the flow of materials drawn therefrom.

Alternatively, as further described below, one scope may include an electrode and an electrical insulating sheath for passage of an electrical current along the selected scope to the distal scope end for use during electro-surgery. In this arrangement, current passes from the active electrode to the surgical site along the scope, separated from the patient by the insulating sheathing. In this manner, the electrical properties of the electrode may be adjusted for cutting, dessication or coagulation between the electrode and the surgical site of interest. During, prior or at the conclusion of the electro-surgery operation, the fluid management apparatus may be operated to provide suction and irrigation to wash the surgical site and/or wet the tissue to reduce contact impedance.

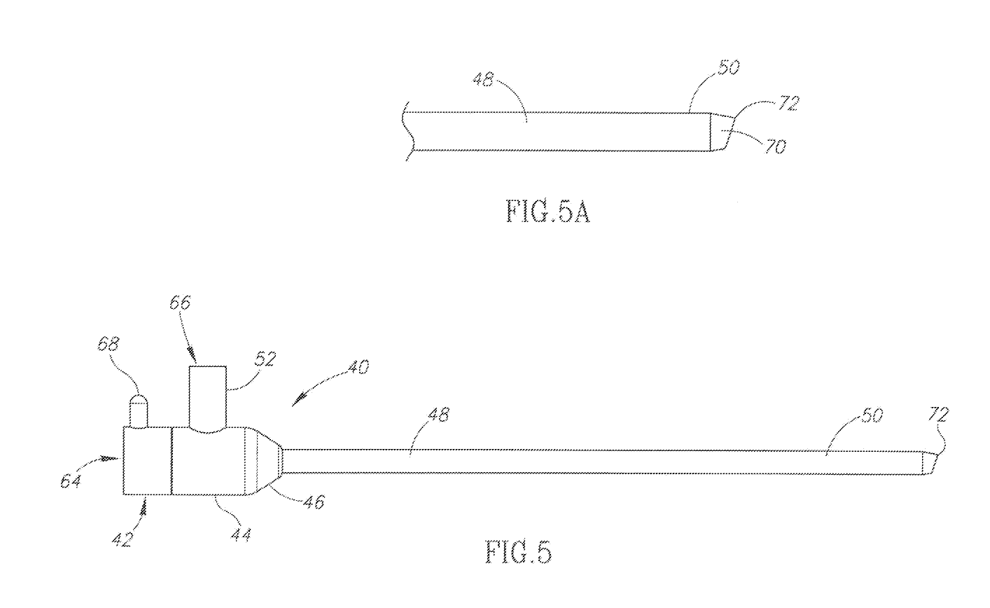

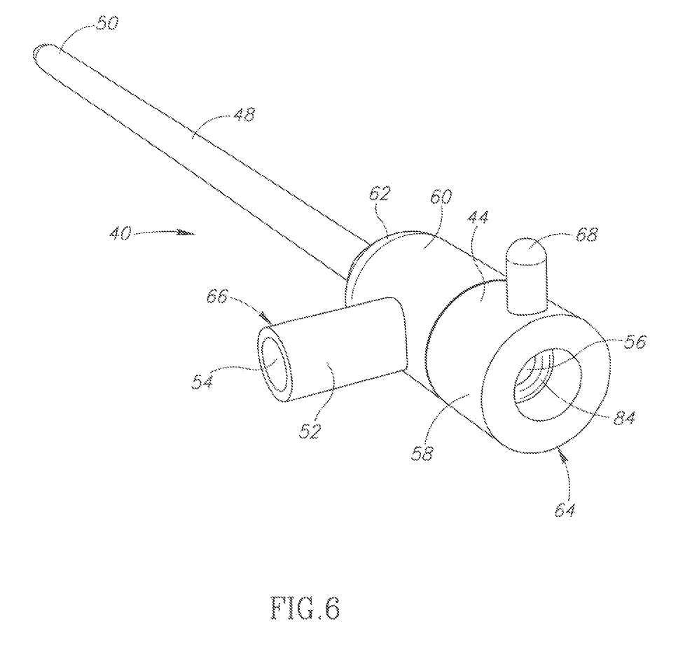

FIGS. 5-7 illustrate an embodiment of an improved trephine 40 according to the present invention. The illustrated trephine 40 includes a trephine body 42 formed of a cylindrical section 44 converging in a conical section 46 to a cannulated rod, cannula, or tube 48, terminating in a tip section 50. The trephine body 42 includes a lateral or side port 52 having a side port passage 54 which communicates with a main passage 56 of the trephine body 42. The illustrated cylindrical section 44 is further divided into a rear or proximal section 58, a center section 60, and a front or distal section 62. The proximal section 58, distal section 62, and the cannula 48 form a body assembly 64 of the trephine 40. The center section 60 and the side port 52 form a side port assembly 66. The body assembly 64 is provided with a radially projecting post 68, as on the proximal section 58.

Referring to FIG. 5A, the illustrated tip section 50 includes a conically tapered termination 70 which is cut at an angle to form a projection cannula tip 72. The conical taper of the termination 70 facilitates movement of the tip section 50 through an incision and past tissues and organs with less injury thereto in comparison to a cylindrical termination of the cannula 48. Because the cannula tip 72 projects outwardly, to further reduce injury, it is important for the surgeon to know its orientation. In the illustrated trephine 40, the post 68 is aligned with the cannula tip 72 to provide the surgeon with a visual indication of the angular orientation of the cannula tip 72 with respect to the side port 52.

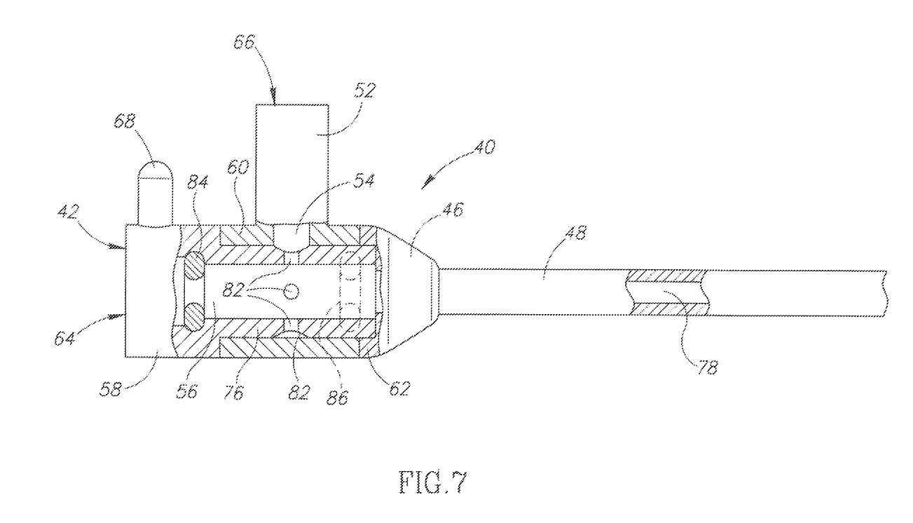

The side port assembly 66 is rotatably mounted on the body assembly 64 for relative rotation therebetween. FIG. 7 illustrates an exemplary configuration of the manner in which the side port assembly 66 is rotatably mounted with respect to the body assembly 64. In the illustrated trephine 40, the center 60 of the trephine body 42 is an annular member or ring from which the side port 52 projects. The body assembly 64 includes a cylindrical neck member or neck 76 which extends from the proximal section 58 and is sized to extend through the center 60 to the distal section 62 of the trephine body 42 into which it is secured, as by being press fit or the like. It is foreseen that the neck 76 could alternatively extend from the distal section 62 toward and be secured to the proximal section 58. It is also foreseen that the neck 76 could be a separate part and be secured to both the proximal section 58 and the distal section 62. In any case, the proximal section 58, the neck 76, and the distal section 62, along with the cannula 48 form an assembled unit, the body assembly 64.

The illustrated neck 76 includes a portion of the main passage 56 which communicates with a cannula passage 78 through the cannula 48. The side port passage 54 communicates with the main passage 56. In the illustrated trephine 40, the neck 76 is provided with an external circumferential groove 80. A plurality of apertures or holes 82 are formed through the wall of the neck 76 within the groove 80. The groove 80 and holes 82 cooperate to provide fluid communication between the side port passage 54 and the main passage 56 for any angular relation between the body assembly 64 and the side port assembly 66.

The illustrated trephine 40 includes an O-ring 84 positioned in the main passage 56 and may include a second or distal O-ring 86 positioned at a location distally spaced from the O-ring 84. It is foreseen that the second O-ring 86 could be positioned between the first or proximal O-ring 84 and the side port 52 or, as illustrated, between the side port 52 and the distal body section 62. The proximal O-ring 84 is preferably formed of an elastomeric material and provides a cushion for an instrument (not shown) extending through the trephine 40 and a fluid seal to prevent fluid flow out the rear of the trephine 40.

The distal O-ring 86 provides balance for an instrument extending through the trephine 40 and loosely engages such an instrument such that it does not prevent fluid flow past its area of engagement. Alternatively, it is foreseen that an inner surface of the O-ring 86 could be notched or otherwise shaped to enable fluid flow past it. This allows the trephine 40 to dissipate pressure within the surgical site and reduces tension at the incision or within soft tissue when the instrument is being manipulated in soft tissue. The loose engagement of the distal O-ring 86 and the engagement between the received surgical instrument and the O-ring 84 allows for maneuverability of the received instrument within the trephine 40, providing a range of access to the surgical site greater than the corresponding diameter of the cannula passage 48. In this way, the maneuverability of the received instrument resembles plastic deformation in that the angular movement of the instrument associated with the tip section 50 is greater than the angular movement of the body assembly 64.

Because of the use of the O-rings 84 and 86 and because of deep engaging surfaces of the body assembly 64 and the side port assembly 66, the trephine 40 is maintained as a single use instrument eliminating the potential bio-hazards associated with reused surgical instruments. Based upon the illustrated configuration, it is difficult to reliably sterilize the trephine 40 after use within a patient. While the proximal O-ring 84 could be removed for autoclaving and replaced, the distal O-ring 86 is inaccessible. Based upon the illustrated configuration, the instrument must be disposed because small amounts of tissue from the patient and any pathogens associated therewith may enter the interfaces between the components of the assemblies 64 and 66. For these reasons, the trephine 40 must be disposed after a single use or recycled in a manner that prevents the release of possible biological hazards.

The trephine 40 may be provided with a means (not shown) of releasably interlocking a surgical instrument extending therethrough to the trephine 40, such as a so-called j-lock or bayonet mechanism.

In use of the trephine 40, an incision is made at an appropriate location, and a guide wire (not shown), such as a nitinol guide wire, is advanced to the surgical site, using radiant imaging, such as fluoroscope, if necessary. The cannula 48 of the trephine 40 is sleeved onto the guide wire, and the tip section 50 is advanced toward the surgical site. The guide wire can then be removed, and a surgical instrument is inserted through the trephine 40 toward the surgical site. If required, the surgical site can be triangulated with a second endoscopic instrument for direct viewing, assistance with the surgical procedure occurring through the trephine 40, fluid management at the surgical site, or the like.

As previously discussed, in one embodiment of the trephine 40, the body assembly 64 may be configured for receiving an electrode with an elongated portion extending through the cannula passage 78, the elongated portion being coated or otherwise insulated (not shown) for use as an electro-cauterization device connected to the trephine 40, the elongated portion having a non-insulated tip for selectively cauterizing tissues at the surgical site.

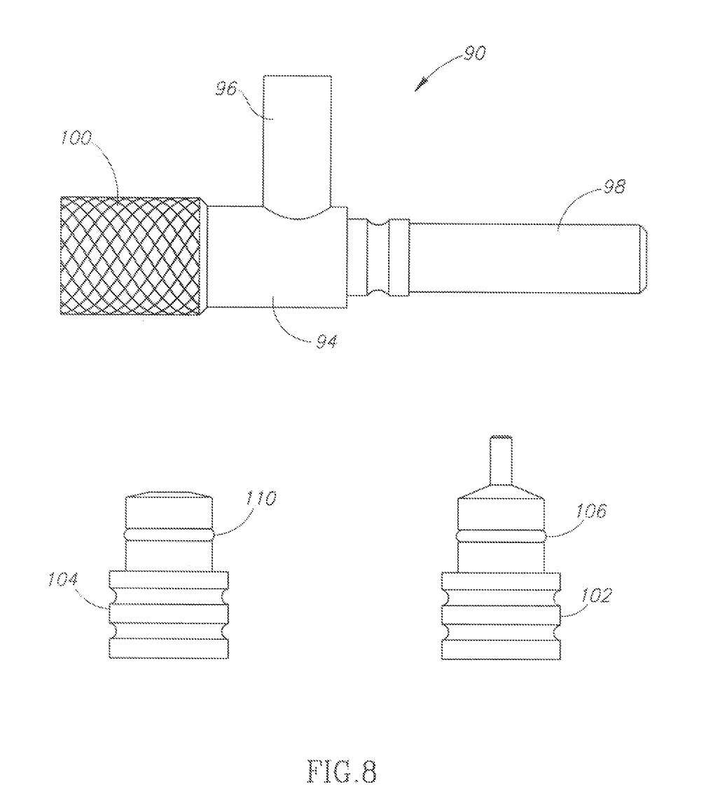

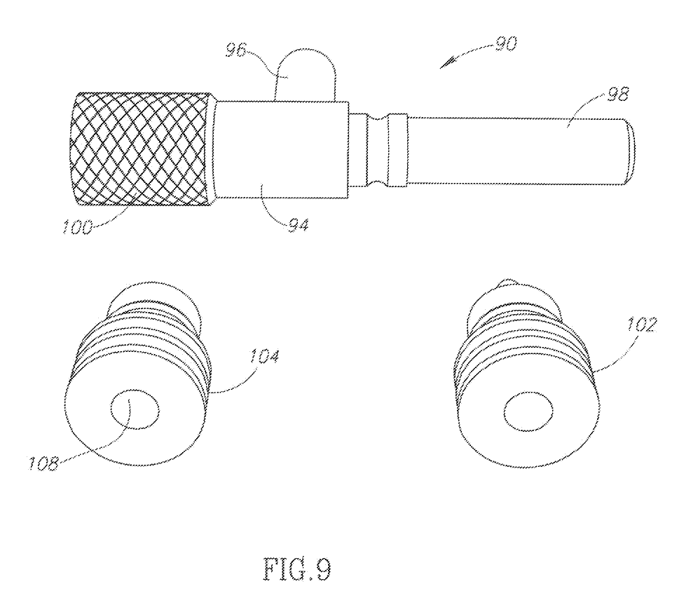

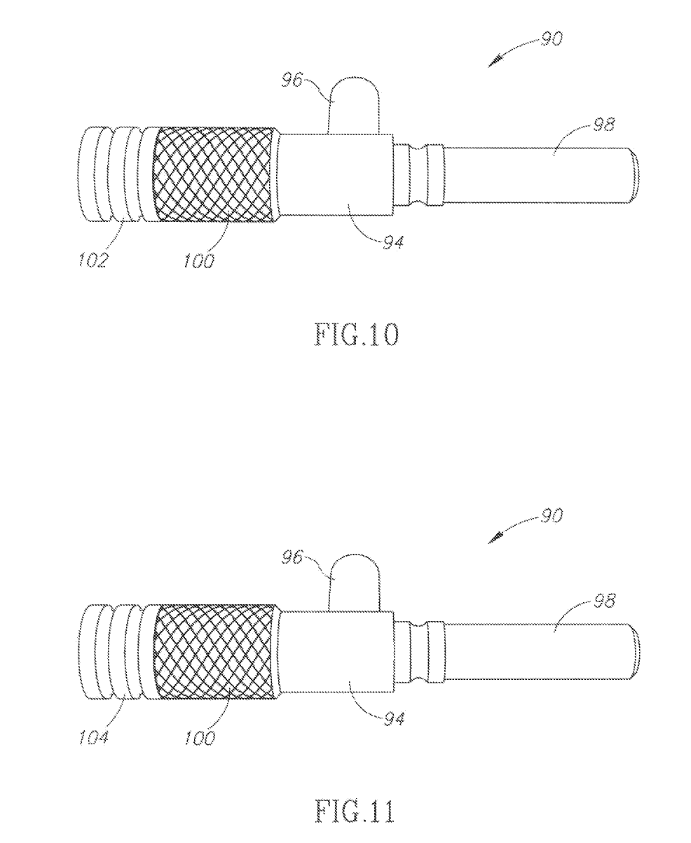

FIGS. 8-14 illustrate an embodiment of an improved outflow or egress adapter 90 according to the present invention. Generally, the adapter 90 is used with a second endoscopic instrument 92 to prevent or control the outflow of fluids from a surgical site or to enable the insertion of surgical instruments (not shown) through the adapter 90 and endoscope 92. The illustrated adapter 90 includes a main body section 94 having an axial main passage (not shown) therethrough and having a side port 96 projecting radially therefrom. The side port 96 has an axial passage (not shown) extending therethrough and communicating with the main passage of the main body 94. The adapter 90 includes a distal end port 98 sized for reception in the second endoscopic instrument 92. The adapter 90 has a proximal port 100 sized to receive either a closed plug 102 or an open or cannulated plug 104. The illustrated proximal port 100 has a knurled external surface for positive gripping by the surgeon. The closed plug 102 is inserted into the proximal port 100 to prevent the outflow of fluids from the adapter 90. A closed plug O-ring 106 is secured to the closed plug 102 to positively seal the closed plug within the proximal port 100. In contrast, the cannulated plug 104 has an axial passage 108 extending therethrough for receiving surgical instruments or for the controlled outflow of fluids from the adapter 90. The cannulated plug 104 may be provided with an O-ring 110, as illustrated in FIG. 8. FIG. 10 shows the outflow adapter 90 with the closed plug 102 inserted in its proximal end port 100. FIG. 11 shows the outflow adapter 90 with the cannulated plug 104 inserted in its proximal end port 100.

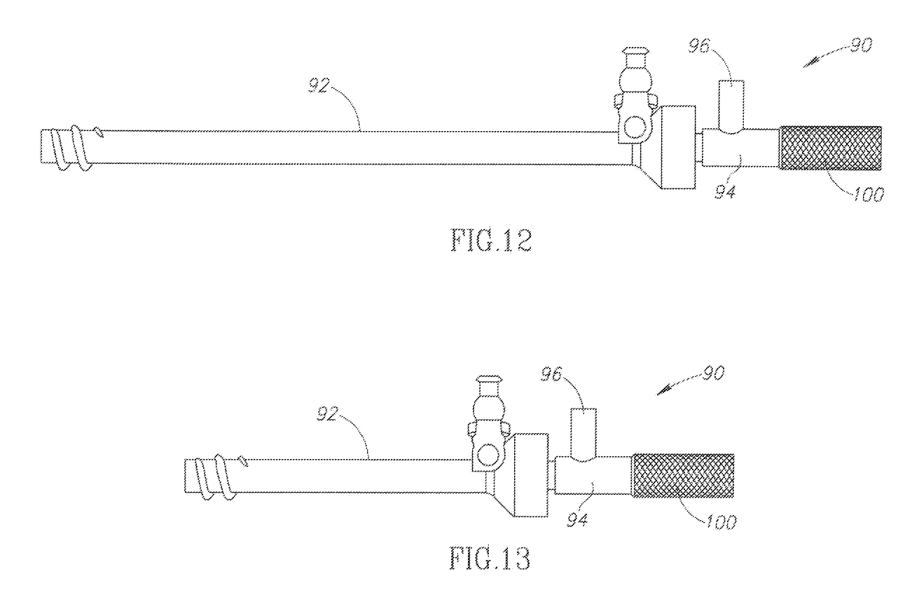

An alternative canulated endoscope instrument 92 is illustrated in FIGS. 12-13 with a flexile cannula located between a second distal end 112 associated with a helical thread 114 and a second proximal end 116 associated with a flexible gasketed material adapted to receive the distal end port 98 of the outflow adapter 90, the second proximal end 116 being generally adapted to restrict the flow of fluid. The cannulated endoscopic instrument 92 also is illustrated with an outflow port 118 for selectively releasing fluid from the cannulated endoscopic instrument 92. Additional access is provided by the side port 96 for suction, irrigation or other surgical devices.

The outflow adapter 90 may be used during triangulation when a second incision is made at an appropriate location, and a guide wire is inserted therethrough and advanced toward the surgical site. The cannulated endoscopic instrument 92 is telescoped over the guide wire and advanced toward the surgical site. An instrument such as an arthroscopic shaver (not shown) can be inserted through the cannulated endoscopic instrument 92 and used, for example, for a debriding procedure. The shaver is then removed, and the outflow adapter 90 is inserted into the instrument 92 to control the outflow of fluids from the surgical site.

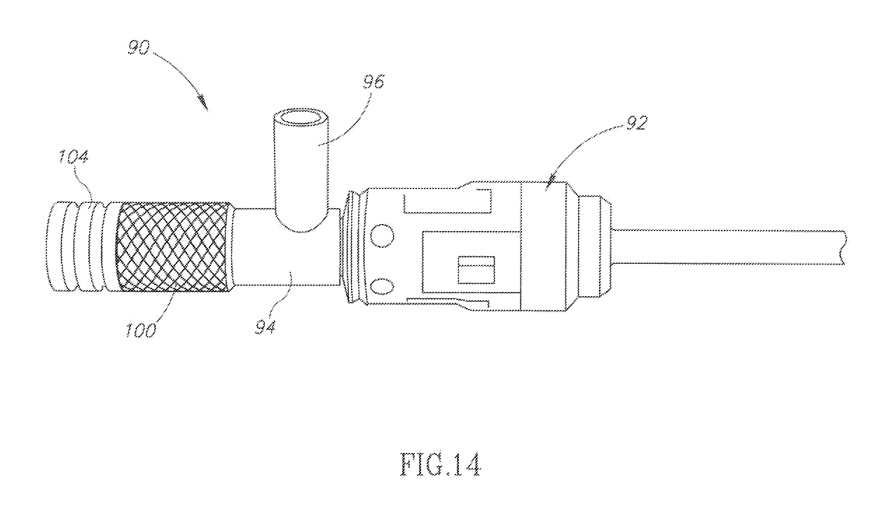

Alternatively, the outflow adapter 90 may be received by one end of a shaver 120 providing access to the surgical site and controlling the outflow of fluid as illustrated in FIG. 14. The closed plug 102 can be used to prevent the outflow of fluids, while the cannulated plug 104 may be used for receiving surgical instruments or for the controlled release of such fluids. The illustrated plugs 102 and 104 are circumferentially ribbed to facilitate gripping by the surgeon to insert the plugs 102 or 104 into the adapter 90 or remove them therefrom.

It is to be understood that while certain forms of the present invention have been illustrated and described herein, it is not to be limited to the specific forms or arrangement of parts described and shown.

What is claimed and desired to be secured by Letters Patent:

1. An improved endoscopic surgery fluid management apparatus for controlling the delivery of liquid to a surgical site during a surgical procedure wherein the fluid management apparatus comprises a plurality of fluid restriction devices and a trumpet valve including a first valve in communication with a second valve along a main passage for selective transmission of fluid to the surgical site, the improvement comprising:
a fluid network operationally connected between said trumpet valve and said restriction devices for fluid communication of said transmitted fluid throughout said fluid network;
a main fluid conduit in communication with a fluid delivery means and associated with the first valve; and
a patient conduit extending between the second valve and an endoscopic instrument for selective transmission of fluid through the endoscopic instrument towards the surgical site.

2. The improved endoscopic surgery fluid management apparatus of claim 1 further comprising a first passage in communication with the first valve and a second passage in communication with the second valve, wherein one of said first passage and second passage is associated with a pneumatic source and the other of said first and second passage is associated with a hydraulic source.

3. The improved endoscopic surgery fluid management apparatus of claim 2 wherein said fluid restrictive devices comprises a first selector in association with said patient conduit a second selector in association with the main fluid conduit and a third selector in association with first valve for selectively delivering fluid at the surgical site.

4. The improved endoscopic surgery fluid management apparatus of claim 1 further comprising a first passage in communication with the first valve and a second passage in communication with the second valve, wherein said second passage is associated with a surgical instrument and said first passage is associated with at least one of a pneumatic source and hydraulic source, wherein said endoscopic instrument and said surgical instrument are angularly oriented in association with the surgical site.

5. The improved endoscopic surgery fluid management apparatus of claim 4 wherein said pneumatic source in connection with one of said endoscopic instrument and said surgical instrument provides simultaneous fluidic communication with said surgical site.

6. A method of using a fluid management apparatus for selectively controlling the fluid at a first surgical site comprising the steps of:
providing a hydraulic source and a pneumatic source in communication with a fluid network comprising a plurality of tubular passages operationally connected to a first surgical instrument associated with the first surgical site;
spacing a trumpet valve and the first surgical instrument in a balanced position in relation to each other and the first surgical site; and
selectively activating a first and second valve associated with said trumpet valve extending between a main conduit and a patient conduit associated with the fluid network, said first valve and said second valve associated with one each of said hydraulic source and said pneumatic source, said patient conduit associated with the first surgical instrument whereby debris is selectively aspirated from the first surgical site.

7. The method of using a fluid management apparatus according to claim 6 further comprising the steps of:
attaching a second surgical instrument to a triangulation scope conduit wherein the second surgical instrument in communication with said first valve and said first surgical instrument is in communication with said patient conduit extending from said second valve;
introducing a second surgical instrument in angularly relation to said first surgical instrument through a second surgical site towards the first surgical site; and
selectively operating said first and said second valves for simultaneous irrigation and debridement of the first surgical site.

8. The method of using a fluid management apparatus according to claim 7 wherein the second surgical instrument includes an electrode and an insulating sheath for passage of an electrical current along the second surgical instrument to a distal end for electrocauterization of said first surgical site.

9. An improved endoscopic surgery fluid management apparatus for controlling the delivery of liquid to a surgical site during a surgical procedure wherein the fluid management apparatus comprises a plurality of fluid restriction devices and a trumpet valve including a first valve in communication with a second valve along a main passage for selective transmission of fluid to the surgical site, the improvement comprising:
a fluid network operationally connected between said trumpet valve and said restriction devices for fluid communication of said transmitted fluid throughout said fluid network;
a main fluid conduit in communication with a fluid delivery means and associated with the first valve; and
a patient conduit extending between the second valve and a surgical instrument for selective transmission of fluid through the endoscopic instrument towards the surgical site.

10. The improved endoscopic surgery fluid management apparatus according to claim 9 wherein said surgical instrument includes an electrode and an insulating sheath for passage of an electrical current along the selected instrument to a distal end for electro-cauterization of said surgical site.

11. The improved endoscopic surgery fluid management apparatus according to claim 9 wherein said surgical instrument includes a trephine body formed of a cylindrical section converging in a conical section to a cannulated rod terminating in a tip section; said trephine body having a lateral port with a side passage in communication with a main passage of the trephine body.

12. The improved endoscopic surgery fluid management apparatus according to claim 11 wherein said trephine body further includes a securing means for releasable receipt of a surgical instrument extending therethrough.

13. The improved endoscopic surgery fluid management apparatus according to claim 9 wherein said surgical instrument is adapted for receipt of an adapter having an axial main passage and a radially projecting side port extending therefrom.
